(19) United States
(12) Patent Application Publication
(10) Pub. No.: US 2009/0156906 A1
(43) Pub. Date: Jun. 18, 2009

(54) Patient-centric data model for research and clinical applications

(76) Inventors: Michael N. Liebman, Kennett Square, PA (US); Richard Mural, Johnstown, PA (US)

Correspondence Address: STEPTOE & JOHNSON LLP, 1330 CONNECTICUT AVENUE, N.W., WASHINGTON, DC, 20036, US

(21) Appl. No.: 12/145,840

(22) Filed: Jun. 25, 2008

Related U.S. Application Data
(60) Provisional application No. 60/946,059, filed on Jun. 25, 2007.

(51) Int. Cl. A61B 5/00; G06F 17/30

(52) U.S. Cl. 600/300; 707/104.1; 707/E17.009

(57) ABSTRACT

The invention relates to a federated patient-centric database which is modular and disease agnostic.

### CLAIM OF PRIORITY

This application is claims priority to U.S. patent application Ser. No. 60/946,059, filed on Jun. 25, 2007, the entire contents of which are hereby incorporated by reference.

### STATEMENT REGARDING FEDERALLY SPONSORED RESEARCH OR DEVELOPMENT

This invention was made with government support under the Clinical Breast Care Project, Prime Award No. USAMRAA # W81XWH-05-2-0053, Subaward Number 114809, “Patient-Centric Data Mode for Research and Clinical Application,” awarded by the Henry M. Jackson Foundation For the Advancement Of Military Medicine, Inc.

### TECHNICAL FIELD

The invention relates to a patient-centric data model for research and clinical applications, which can be modular and disease agnostic.

### BACKGROUND

Many diseases and disorders, such as cancer, have very complex genetic and phenotypic abnormalities and an unpredictable biological behavior. The cancer cell for example, represents the end-point of successive generations of clonal cell evolution, multiple gene mutations, genomic instability, and erroneous gene expression. The biological behavior of cancer is determined by multiple factors, most importantly the biological characteristics of the individual cancer, but also the biology of the patient such as age, sex, race, genetic constitution and the like, and the location of the cancer. This biological and genetic complexity of cancer means that in any individual, cancer may follow an unpredictable clinical course, with an uncertain outcome for the patient. Where multiple treatment options are available for a particular cancer, it is necessary to have an accurate diagnosis for the patient, so that treatment can be tailored to the individual disease of that patient.

The clinical and information tools currently available to clinicians for the classification and diagnostic evaluation of cancer and other diseases have serious limitations, especially when applied to an individual patient. It would be desirable to create a federated database which integrates clinical and biological databases for a given disease or condition.

### SUMMARY

In one aspect, a method for predicting disease progression or outcome includes storing patient information in a database, storing clinical data in a database, creating a federated database from at least one database selected from the group that includes a patient information database, a clinical database, a genomic database, a proteomic database, an imaging database and a disease database and submitting a request for information. The method can further include generating a patient profile with a prediction on disease progression or outcome. The method can further include generating a treatment plan. The method can further include predicting disease recurrence. The method can further include collecting patient information. The method can further include collecting clinical data.

The clinical database can include predicted genetic risk, biomarkers, tumor heterogeneity, pathology report, pathology images, diagnosis co-morbidities, outcomes, diagnostic images, surgical reports, radiation protocols, chemotherapy protocols, post-therapy co-morbidities, protein expression, gene expression, genotyping, sequencing data and DNA copy number analysis from tissue samples or blood samples of the patient or combinations thereof. The patient information database can include clinical history, family history, reproductive history, gynecologic history, lifestyle exposures or quality of life priorities or combinations thereof. The genomic database can be an Entrez database. The proteomic database can be an Entrez database. The disease can be breast cancer, cervical cancer, endometrial cancer, ovarian cancer or uterine cancer. The disease can be cardiovascular disease. The disease can be diabetes.

The method can further include creating a federated database from a patient information database. The method can further include creating a federated database from a clinical database. The method can further include creating a federated database from a genomic database. The method can further include creating a federated database from a proteomic database. The method can further include creating a federated database from an imaging database. The method can further include creating a federated database from a disease database.

In another aspect, a method for diagnosing breast cancer progression or outcome can include storing patient information in a database, storing clinical data in a database, creating a federated database from at least one database selected from the group that includes a patient information database, a clinical database, a genomic database, a proteomic database, an imaging database and a disease database, and submitting a request for information. The method can further include generating a patient profile with a prediction on breast cancer progression or outcome. The method can further include generating a treatment plan. The method can further include predicting disease recurrence. The method can further include collecting patient information. The method can further include collecting clinical data.

The clinical database can include predicted genetic risk, biomarkers, tumor heterogeneity, pathology report, pathology images, diagnosis co-morbidities, outcomes, diagnostic images, surgical reports, radiation protocols, chemotherapy protocols, post-therapy co-morbidities, protein expression, gene expression, genotyping, sequencing data and DNA copy number analysis from tissue samples or blood samples of the patient or combinations thereof. The patient information database can include clinical history, family history, reproductive history, gynecologic history, lifestyle exposures or quality of life priorities or combinations thereof. The genomic database can be an Entrez database. The proteomic database can be an Entrez database.

The method can further include creating a federated database from a patient information database. The method can further include creating a federated database from a clinical database. The method can further include creating a federated database from a genomic database. The method can further include creating a federated database from a proteomic database. The method can further include creating a federated database from an imaging database. The method can further include creating a federated database from a disease database.

In a further aspect, a system for predicting disease progression or outcome can include a federated database created from at least one database selected from the group that includes a patient information database, a clinical information database, a genomic database, a proteomic database, an imaging database and a disease database. The clinical database can include predicted genetic risk, biomarkers, tumor heterogeneity, pathology report, pathology images, diagnosis co-morbidities, outcomes, diagnostic images, surgical reports, radiation protocols, chemotherapy protocols, post-therapy co-morbidities, protein expression, gene expression, genotyping, sequencing data and DNA copy number analysis from tissue samples or blood samples of the patient or combinations thereof. The patient information database comprises clinical history, family history, reproductive history, gynecologic history, lifestyle exposures or quality of life priorities or combinations thereof. The genomic database can be an Entrez database. The proteomic database can be an Entrez database.

The details of one or more embodiments are set forth in the accompanying drawings and the description below. Other features, objects, and advantages will be apparent from the description and drawings, and from the claims.

### BRIEF DESCRIPTION OF DRAWINGS

FIG. 1 is a flow diagram illustrating a system for generating models of disease progression or outcome.

FIG. 2 is an illustration depicting hierarchies.

FIG. 3 is an illustration depicting a physician's workflow.

FIG. 4 is an illustration depicting a workflow based physician-patient process.

FIG. 5 is an illustration depicting patient-modeling.

FIG. 6 is an illustration depicting stratification of patient populations.

FIG. 7 is an illustration depicting a search repository.

FIG. 8 is an illustration depicting data fusion and mammography.

FIG. 9 is a flow diagram illustrating analysis of gene expression data using PACE.

FIG. 10 is a screen shot of the Clinical Laboratory Workflow System from Cimarron.

FIG. 11 is a flow diagram of current version of Windber Research Institute's data warehouse content.

FIG. 12 is an illustration of NCR Teradata RDBMS.

FIG. 13 is an illustration of Teradata defined data warehouse schema.

FIG. 14 is an illustration of a research gateway data cube.

FIG. 15 is a screen shot of the Windber Research Institute Data Mart.

FIG. 16 is an illustration of a decision support system.

FIG. 17 is an illustration of a Petri net called Stochastic Activity Networks (SANs).

FIG. 18 is an illustration of a Spotfire output.

FIG. 19 is an illustration of a Bayesian network.

FIG. 20 is a screen shot of LexiMine/SPSS.

### DETAILED DESCRIPTION

Electronic patient records (EHR) and biological databases are currently available. A system which effectively and efficiently assimilates patient information databases, clinical databases, genomic databases, proteomic databases, imaging databases and disease databases into a dynamic system that can be rapidly extended into both research laboratories environments and clinical practice is desirable. Such a system can be portable across all diseases including but not limited to, cardiovascular disease, cancer, diabetes, aging or women's health issues.

In one embodiment, the system can include the federation of patient information and biological databases relating to breast cancer. The system can further include integration of patient information and biological databases relating to other cancers such as breast, prostate, bladder, leukemia, lymphoma, central nervous system, lung, colorectal, melanoma, uterine, renal cell, pancreatic, ovarian, endometrial, cervical or pleural cancers.

The creation of a patient-centric data model that exists as a federated data model that is modular and extensible to be disease agnostic enables the rapid integration of new sources of patient information from clinical, molecular and imaging into a model that abstracts the clinical and molecular perspectives in an object layer that integrates the data elements in a one-to-many mapping. The collection of abstract patient modules, in the object layer, further enables the development of best practice approaches to each area of clinical and molecular focus and their subsequent mapping into a workflow-based physician-patient process for enhanced diagnosis, decision-making and treatment of patients in a collaborative manner. This approach further redefines translational medicine in a manner that emphasizes the need to define problems in a clinical environment that can be brought to the laboratory for research with the subsequent conversion of research results into immediate clinical utility.

### Database Sources

Examples of databases to be federated into a single federated database can include patient information databases, clinical databases, genomic databases, proteomic databases, imaging databases or disease databases.

Patient information databases can be created from information obtained from questionnaires filled by patients at a clinic or any health care setting. Examples of patient information can include clinical history, family history, reproductive history, gynecologic history, lifestyle exposures and quality of life priorities. Patient information can optionally contain information such as medication being taken by the patient, medical history, occupational information, hobbies of the patient, diet, normal exercise routines, age and sex. More specific examples of information can include whether the patient is undergoing hormone replacement therapy, whether the patient is a drinker or a smoker, whether the patient is regularly exposed to the sun, the geographical location of the patient's residence, whether the patient exercises, and whether the patient is post or pre-menopausal. Patient information can be collected during the patient's first visit and updated during subsequent visits.

A clinical database can include clinical data on predicted genetic risk, biomarkers, tumor heterogeneity, pathology report, pathology images, diagnosis co-morbidities, outcomes, diagnostic images, surgical reports, radiation protocols, chemotherapy protocols and post-therapy co-morbidities. A clinical database can also include experimental data. Experimental data can include protein expression, gene expression, genotyping, sequencing data and DNA copy number analysis from tissue samples and blood samples of the patient. In some diseases or conditions, proteins can be present in body fluids at evaluated levels compared to individuals without malignant disease, and can be sufficiently stable to enable immunodetection. Biological samples such as tissue, serum, lymph, body fluid samples can be collected from patients and analyzed. Sample preparation and purification can be tracked. Body fluids can include blood, urine, sputum, semen, gastric fluids and stool. Data can be acquired under a single set protocol and reviewed by a single pathologist. Where such body fluids are not useful, biopsies of suspect tissues may be used. Overexpression or underexpression can also be detected by either nucleic add detection or protein detection techniques in fluids if they contain cells, or cell lysates that can be released from suspect tissues.

Protein expression data can be generated using 2D-Difference Gel Electrophoresis and Mass Spectrometry (DIGE/MS) technology. Laser capture microdissection (LCM) can also be used to examine protein and gene expression in different cell populations. Alternatively, proteins of interest can be detected in body fluids with immuno-detection techniques using monoclonal or polyclonal antibodies raised against either whole proteins or peptides of interest. Immunodetection techniques can include ELISA/EIA radioimmunoassay, nephelometry, immunoturbidometric assays, chemiluminescence, immunofluorescence (by microscopy or flow cytometry), immunohistochemistry and Western blotting. It can be readily appreciated that other methods for detecting proteins can be used.

High throughput experimental data such as gene expression data of a particular tumor can be generated by using the GE Healthcare CodeLink which utilizes a wide range of pre-arrayed oligonucleotide bioarrays. For example, mRNA expression levels in diseased breast tissue or blood samples can be compared with mRNA expression levels in control breast tissue or blood samples to identify biomarkers and build predictive models of disease progression. The data generated by CodeLink can be correlated by RNA levels measured using a Boehringer system based on RT-PCR. Gene sequencing data can be obtained using the Mega BACE DNA analysis systems. Genotyping data can be generated using the MegaBACE platform from GE Healthcare and can include one or more single nucleotide polymorphisms (“SNPs”) in the DNA of the patient. DNA copy number analysis can be performed using the array comparative genomic hybridization (CGH array system) technique from GenoSensor Array 300 from Vysis. Imaging data can be obtained using for example, mammography, magnetic resonance imaging (MRI), ultrasound, positron emission tomography (PET) and computed tomography (cat scans).

Genomic and proteomic databases can include public domain databases such as Entrez, UniProt, Gene Ontology, Gene, RefSeq. Other public domain databases can include SwissProt, SRS, PDB, KEGG, HUGO and GO.

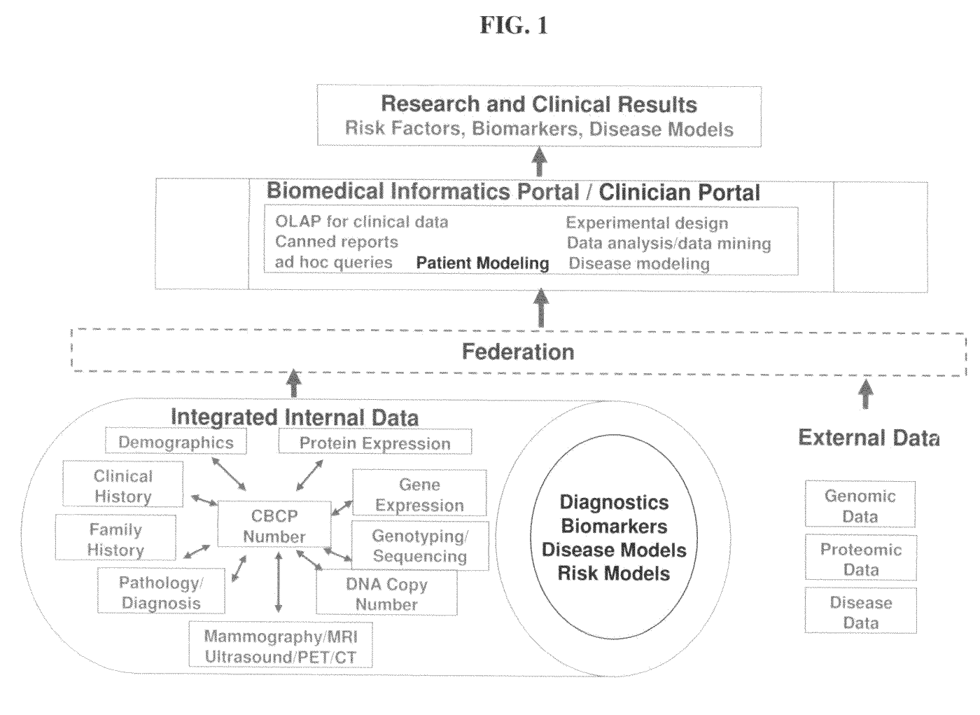

By way of example, FIG. 1 depicts a flow diagram of a system for generating models of disease progression or outcome. Integrated internal data can include data obtained from patient information such as demographics, clinical history, family history, pathology, diagnosis, mammography, MRI, ultrasound, PET, CT, DNA copy number, genotyping, sequencing, gene expression and protein expression. External data can be drawn from public domain databases that includes genomic data, proteomic data and disease data. Both the integrated internal data and external data are federated into one single database. A Bioinformatics Portal or a Clinician Portal can be created based on the federated database. Such portals can include On Line Analytical Processing (OLAP) for clinical data, canned reports, ad hoc queries, patient modeling, experimental design, data analysis, data mining and/or disease modeling to generate research and clinical results.

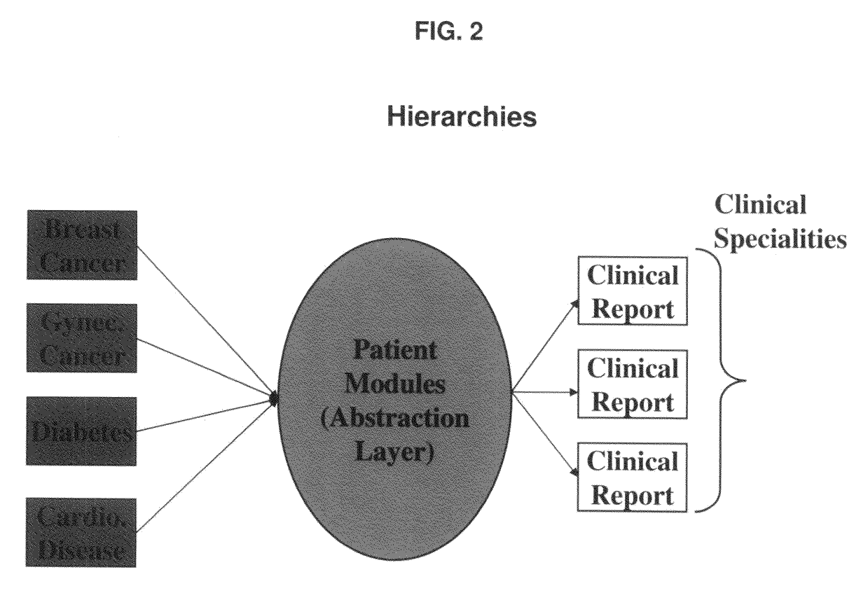

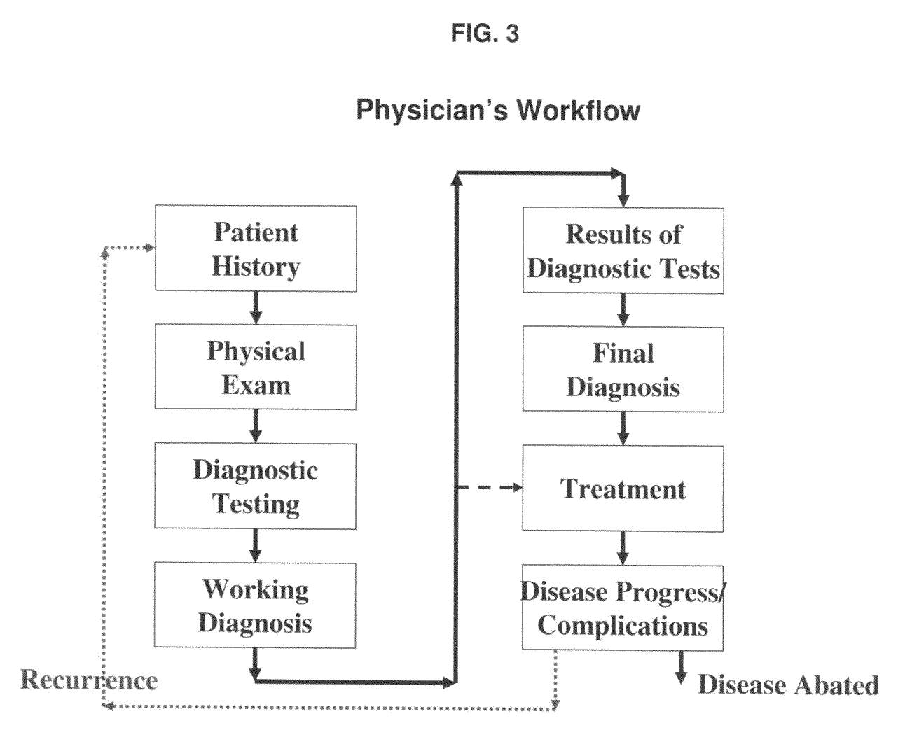

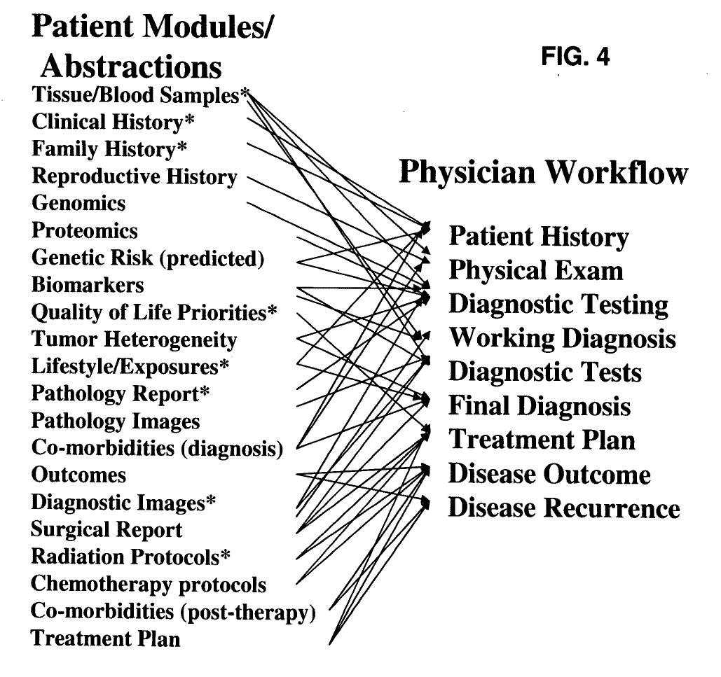

The federated database can enable the rapid integration of new sources of patient information from clinical, molecular and imaging data into a data model that abstracts such data in an object layer. See FIG. 2. The object layer can integrate the data elements into a one-to-many mapping. Patient modules can include data abstraction, clinical report format and/or best practices. The data sources can be mapped into modules and the modules can be mapped into a workflow, e.g. a physician's workflow. See FIGS. 3 and 4.

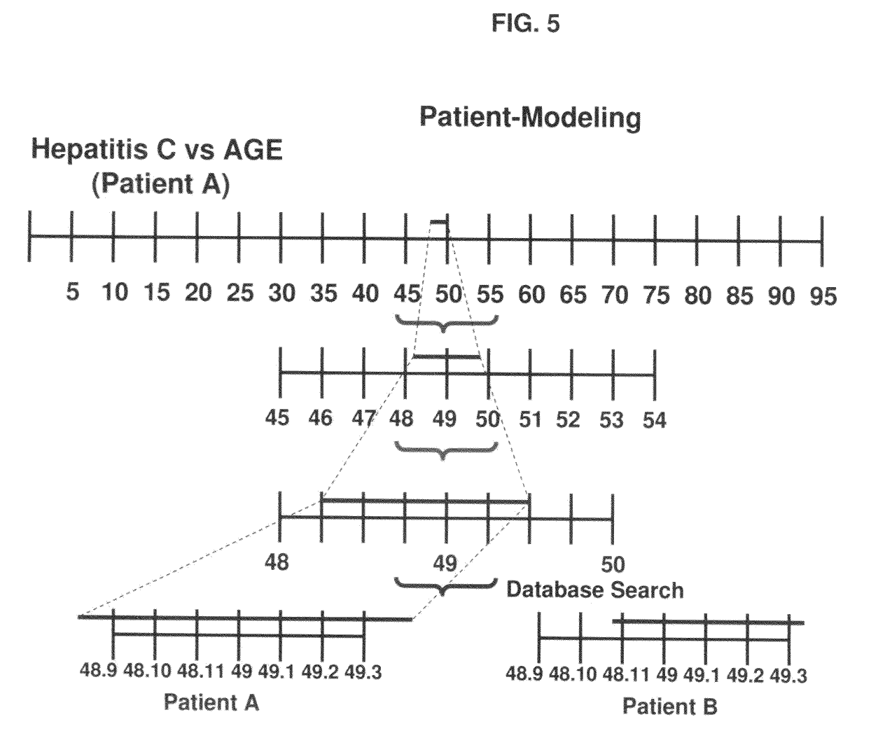

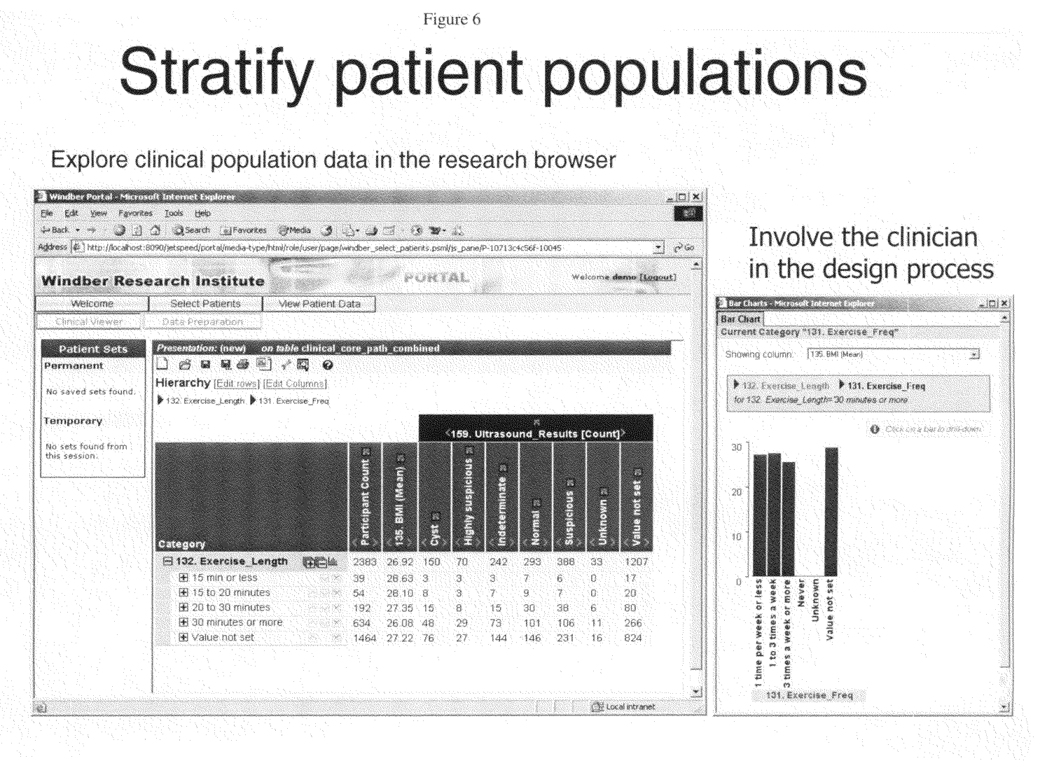

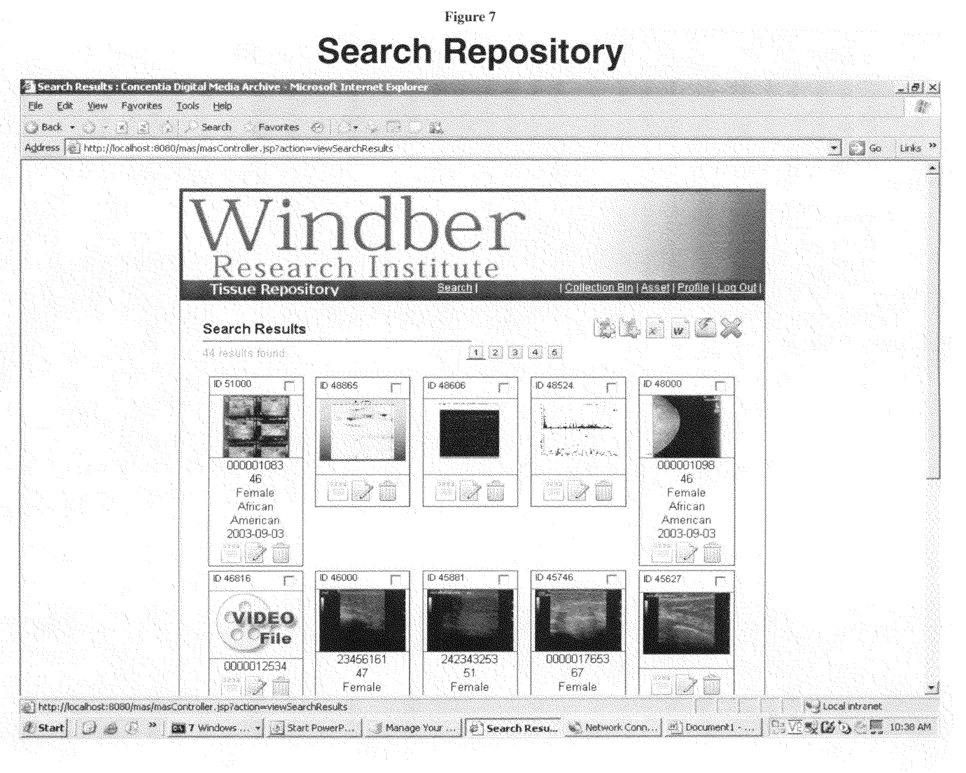

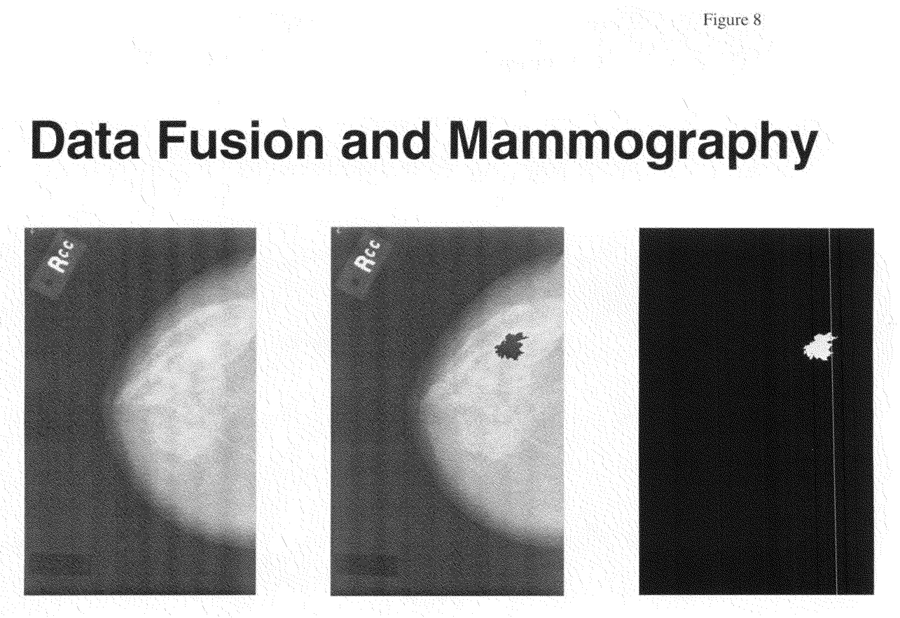

Predictive models of disease progression and outcome can be generated from the federated database using statistical data analysis, predictive modeling, patient population stratification and disease modeling tools. See for example, FIGS. 5 and 6. A search repository can be created. See FIG. 7. Predictive models of disease progression and outcome can also be generated through data fusion and imaging data. See for example, FIG. 8. Such predictive models can be used to power a decision support system that for use by a clinician or a research scientist. Disease modeling can also be achieved using Petri net tool set which is a modeling technology tailored for representing and simulating concurrent dynamic systems from the University of Illinois (http://www.mobius.uiuc.edu/index.html). The analysis of a federated database can be used to generate a treatment protocol or predict disease recurrence, progression or outcome. The federated database can also be used to identify disease or potential disease or risk of disease in people who do not yet have any signs of disease or at least have no significant outward signs of disease. Additionally, the federated database can be used to generate multiple diagnoses or to generate predictions about the likelihood of diagnosis based on evidence of other diagnosis. The federated database can also be used for textmining and extracting molecular events and changes associated for example, with breast development and breast disease through a collection of journal articles, preprocessing of collected text, construction of dictionaries, compilation of patterns, information extraction (NLP) and incorporation of Medline information.

The various techniques, methods, and systems described above can be implemented in part or in whole using computer-based systems and methods. Additionally, computer-based systems and methods can be used to augment or enhance the functionality described above, increase the speed at which the functions can be performed, and provide additional features and aspects as a part of or in addition to those described elsewhere in this document. Various computer-based systems, methods and implementations in accordance with the above-described technology are presented below.

In one implementation, a general-purpose computer can have an internal or external memory for storing data and programs such as an operating system (e.g., DOS, Windows 2000™, Windows XP™, Windows NT™, OS/2, UNIX or Linux) and one or more application programs. Examples of application programs include computer programs implementing the techniques described herein, authoring applications (e.g., word processing programs, database programs, spreadsheet programs, or graphics programs) capable of generating documents or other electronic content; client applications (e.g., an Internet Service Provider (ISP) client, an e-mail client, or an instant messaging (IM) client) capable of communicating with other computer users, accessing various computer resources, and viewing, creating, or otherwise manipulating electronic content; and browser applications (e.g., Microsoft's Internet Explorer) capable of rendering standard Internet content and other content formatted according to standard protocols such as the Hypertext Transfer Protocol (HTTP). Applications for federating databases include the InforSense software.

One or more of the application programs can be installed on the internal or external storage of the general-purpose computer. Alternatively, in another implementation, application programs can be externally stored in and/or performed by one or more device(s) external to the general-purpose computer.

The general-purpose computer includes a central processing unit (CPU) for executing instructions in response to commands, and a communication device for sending and receiving data. One example of the communication device is a modem. Other examples include a transceiver, a communication card, a satellite dish, an antenna, a network adapter, or some other mechanism capable of transmitting and receiving data over a communications link through a wired or wireless data pathway.

The general-purpose computer can include an input/output interface that enables wired or wireless connection to various peripheral devices. Examples of peripheral devices include, but are not limited to, a mouse, a mobile phone, a personal digital assistant (PDA), a keyboard, a display monitor with or without a touch screen input, and an audiovisual input device. In another implementation, the peripheral devices can themselves include the functionality of the general-purpose computer. For example, the mobile phone or the PDA can include computing and networking capabilities and function as a general purpose computer by accessing the delivery network and communicating with other computer systems. Examples of a delivery network include the Internet, the World Wide Web, WANs, LANs, analog or digital wired and wireless telephone networks (e.g., Public Switched Telephone Network (PSTN), Integrated Services Digital Network (ISDN), and Digital Subscriber Line (xDSL)), radio, television, cable, or satellite systems, and other delivery mechanisms for carrying data. A communications link can include communication pathways that enable communications through one or more delivery networks.

In one implementation, a processor-based system (e.g., a general-purpose computer) can include a main memory, preferably random access memory (RAM), and can also include a secondary memory. The secondary memory can include, for example, a hard disk drive and/or a removable storage drive, representing a floppy disk drive, a magnetic tape drive, an optical disk drive, etc. The removable storage drive reads from and/or writes to a removable storage medium. A removable storage medium can include a floppy disk, magnetic tape, optical disk, etc., which can be removed from the storage drive used to perform read and write operations. As will be appreciated, the removable storage medium can include computer software and/or data.

In alternative embodiments, the secondary memory can include other similar means for allowing computer programs or other instructions to be loaded into a computer system. Such means can include, for example, a removable storage unit and an interface. Examples of such can include a program cartridge and cartridge interface (such as the found in video game devices), a removable memory chip (such as an EPROM or PROM) and associated socket, and other removable storage units and interfaces, which allow software and data to be transferred from the removable storage unit to the computer system.

In one embodiment, the computer system can also include a communications interface that allows software and data to be transferred between computer system and external devices. Examples of communications interfaces can include a modem, a network interface (such as, for example, an Ethernet card), a communications port, and a PCMCIA slot and card. Software and data transferred via a communications interface are in the form of signals, which can be electronic, electromagnetic, optical or other signals capable of being received by a communications interface. These signals are provided to communications interface via a channel capable of carrying signals and can be implemented using a wireless medium, wire or cable, fiber optics or other communications medium. Some examples of a channel can include a phone line, a cellular phone link, an RF link, a network interface, and other suitable communications channels.

In this document, the terms “computer program medium” and “computer usable medium” are generally used to refer to media such as a removable storage device, a disk capable of installation in a disk drive, and signals on a channel. These computer program products provide software or program instructions to a computer system.

Computer programs (also called computer control logic) are stored in the main memory and/or secondary memory. Computer programs can also be received via a communications interface. Such computer programs, when executed, enable the computer system to perform the features as discussed herein. In particular, the computer programs, when executed, enable the processor to perform the described techniques. Accordingly, such computer programs represent controllers of the computer system.

In an embodiment where the elements are implemented using software, the software can be stored in, or transmitted via, a computer program product and loaded into a computer system using, for example, a removable storage drive, hard drive or communications interface. The control logic (software), when executed by the processor, causes the processor to perform the functions of the techniques described herein.

In another embodiment, the elements are implemented primarily in hardware using, for example, hardware components such as PAL (Programmable Array Logic) devices, application specific integrated circuits (ASICs), or other suitable hardware components. Implementation of a hardware state machine so as to perform the functions described herein will be apparent to a person skilled in the relevant art(s). In yet another embodiment, elements are implanted using a combination of both hardware and software.

In another embodiment, the computer-based methods can be accessed or implemented over the World Wide Web by providing access via a Web Page to the methods described herein. Accordingly, the Web Page is identified by a Universal Resource Locator (URL). The URL denotes both the server and the particular file or page on the server. In this embodiment, it is envisioned that a client computer system interacts with a browser to select a particular URL, which in turn causes the browser to send a request for that URL or page to the server identified in the URL. Typically the server responds to the request by retrieving the requested page and transmitting the data for that page back to the requesting client computer system (the client/server interaction is typically performed in accordance with the hypertext transport protocol or HTTP). The selected page is then displayed to the user on the client's display screen. The client can then cause the server containing a computer program to launch an application to, for example, perform an analysis according to the described techniques. In another implementation, the server can download an application to be run on the client to perform an analysis according to the described techniques.

### EXAMPLES

### Clinical Data

The source of data will be clinical data generated by the Windber/Walter Reed Medical Clinical Breast Care Project. Currently, >14,000 samples (tissue, serum, lymph) with 10,000 patients/year involved in the program. For data quality, all data was acquired under a single protocol and reviewed by a single pathologist. Clinical operations were carried out by Walter Reed Army Medical Center (WRAMC) and the Joyce Murtha Care Center (JMBCC), along with several other military and civilian medical institutions.

Over 500 data fields exist per patient and these are collected from four questionnaires.

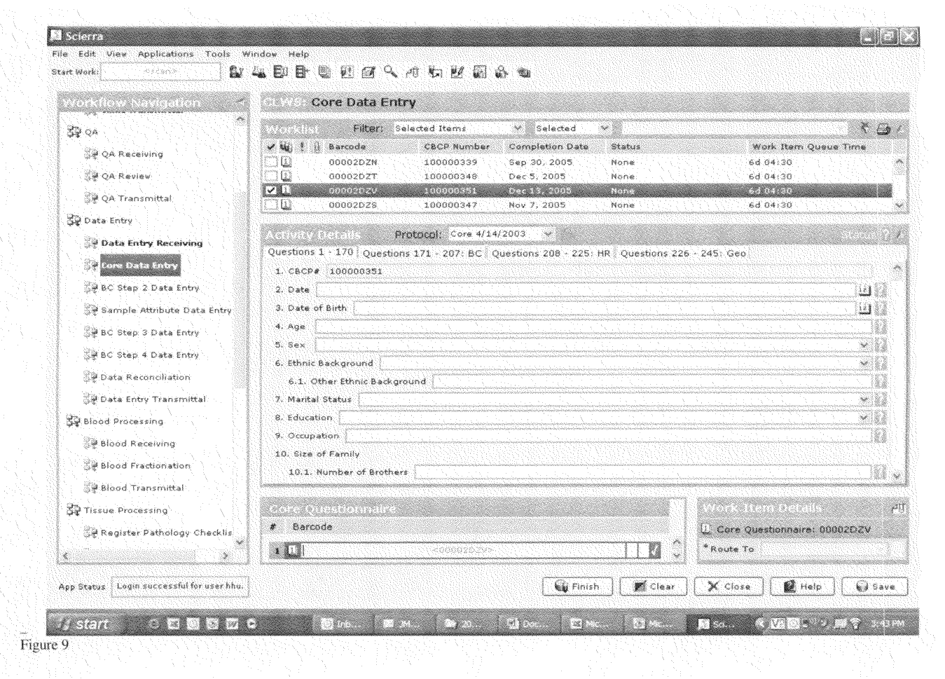

The schema of this Oracle database is hard to understand and nearly impossible to query on a routine basis. CLWS is used solely for tracking not analysis. See FIG. 9. There might be a requirement for KDE integration with CLWS (either at intermediate steps along the data entry WE or just at the end of the process) although the priority is for KDE to interact with the redesigned DW (see later). Data entered via CLWS can not be modified although the preference is that the data should be able to be modified as long as detailed audit trail is captured. All clinical data is entered by this route except the image data which is composed of mammograms, 4d-ultrasound, PET/CT and 3T MRI. This image data is held separately on bespoke hardware and needs to be at least referenced in the redesigned DW

### High Throughput Experimental Data

Sample preparation, purification AND results for all experimental approaches are tracked using the Scierra LWS from Cimarron.

### Gene Expression

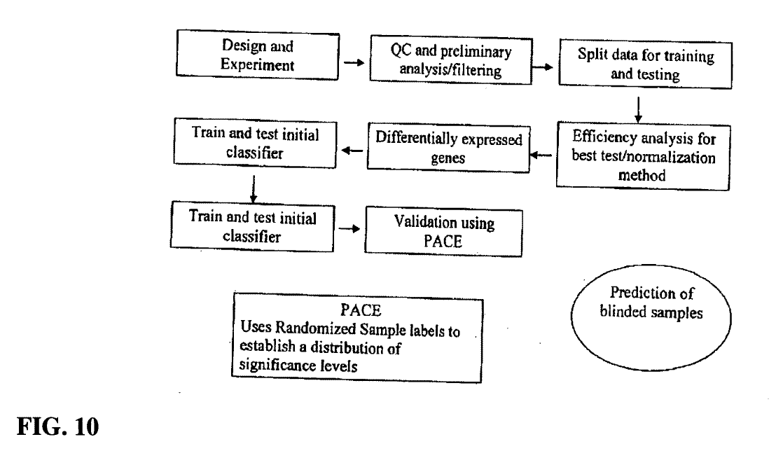

Gene expression data is generated by using the GE Healthcare CodeLink system (pre-arrayed oligonucleotide chips). Typical experiments involve comparing mRNA expression levels between diseased breast tissue/blood samples with controls in order to identify biomarkers and build predictive models of disease progression. A Boehringer system based on RT-PCR is used to assess RNA levels and cross correlate this lower throughput approach with the CodeLink output. See FIG. 10.

### Proteomics

Protein expression data is generated using the 2D-DIGE/MS technology. Accuracy of protein identification is determined using a variety of filters before any downstream annotation and biological interpretation. Laser capture micro dissection (LCM) is also used to examine protein (and gene) expression in different cell populations

### DNA Sequencing

Sequencing data is generated using the MegaBACE platform from GE Healthcare

### Genotyping

Genotype data is generated currently also using the MegaBACE platform from GE Healthcare and Affymetrix machines for SNP genotyping using the 100K chips.

### DNA Copy Number

DNA copy number analysis is carried out using the array comparative genomic hybridization (a-CGH) technique. The machine is from GenoSensor Array 300 from Vysis

### Data Warehouse

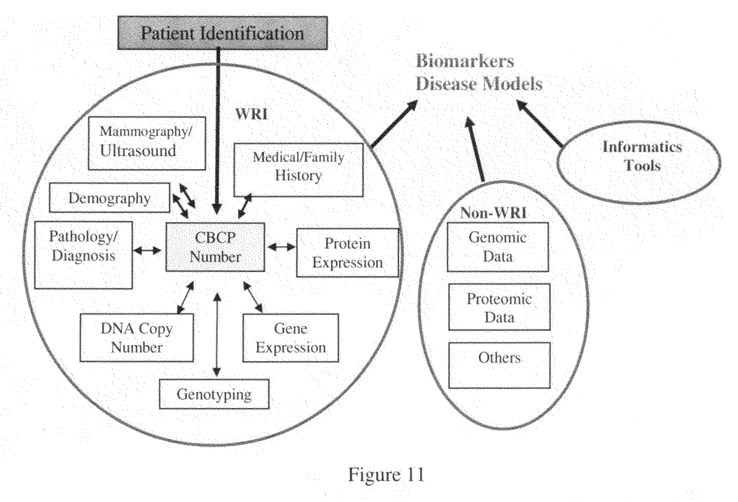

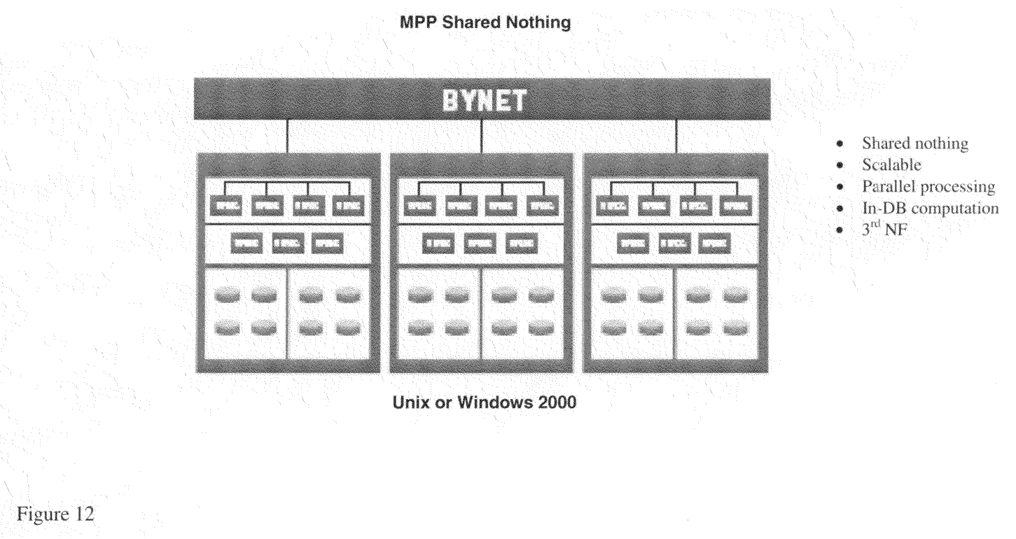

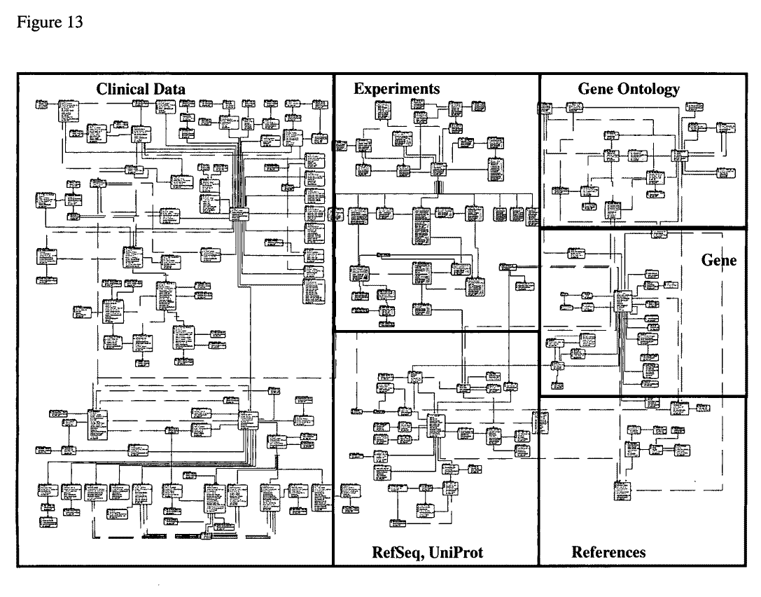

For the last couple of years, WRI have been building a D W to hold all the above clinical and experimental data. WRI decided to take a DW approach because of envisaged limitations using databases when on-line transaction processing involves very large data sets and complex queries. See FIG. 11. NCR Teradata RDBMS has a shared-nothing structure and stores data in third Normal Form with no repeating groups, derived data or optional columns. This DW environment automatically distributes data and balances workloads for parallel processing. See FIG. 12. The current Teradata defined DW schema is separated into 5 modules. See FIG. 13.

On Teradata's recommendation, they adopted a hybrid approach of integration and federation. However, they did integrate some public domain databases (e.g. RefSeq, UniProt, Gene Ontology and Gene). The 3 criteria they used to select the public databases to integrate are maturity, acceptability and essentiality. For the future, they are suggesting that all internal data (which is under their direct control) is integrated in the DW whereas all external data (which they cannot control) is federated. We clearly can help here although our web service plugin would need some modifications since NCBI WSDL is extremely complex.

Some of the current frustrations with the existing Teradata DW include:
1) Data still not in the DW both internal and external sources
2) System still seems unable to cope with the complexity of the queries
3) Incorporated public domain data is proving difficult to maintain
4) Teradata RDBMS has no existing visualization or analytical tools to support their research so feels like data locked in DW with no easy way to mine it!
5) Performance OK but not great—almost every data access demands denormalisation from the 3'd NF

Re the current size of the D W nobody could give me an accurate figure—but many thousands of patients enrolled (or to be enrolled) with 500+ clinical fields, multiple visits per year, each visit resulting in microarray/proteomics/image data—it has to be big.

Re the use of medical image data, WRI see this as a key component currently not addressed in the DW. Current thinking is that these images would be referenced in the DW and the actual images will be held centrally on designated hardware. First step is to collect these images into a central repository (maybe Oracle). They are trying to form a clinical network using some new high speed fiber connection to link together a variety of east coast medical centers including NCI, NIH, John Hopkins, Pitt . . . . Also, may want to apply a similar approach for images generated from proteomics.

### Data Analysis

As previously mentioned, this area is very much under developed due to the shortage of applications that can sit on top of the Teradata D W. Clearly, this will be very different when we have redesigned the DW using Oracle technology

### Visualisation

WRI envisage 2 types of user with very different needs/capabilities:
Clinicians—Portal and OLAP technology thought to be ideal here
Research Scientists—Spotfire (some licenses, would need more) in WF context

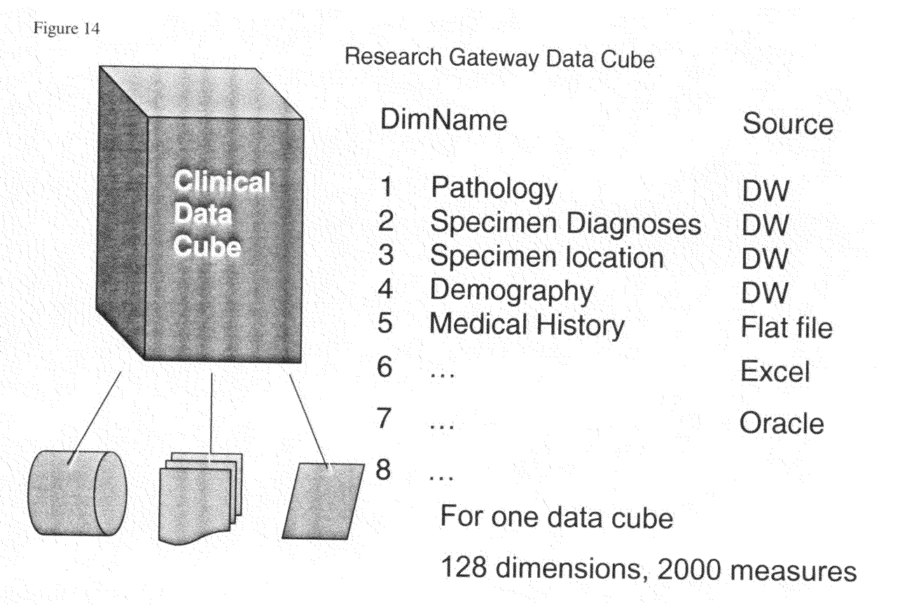

For the clinicians, already put together a ‘Research Gateway based on Portal/OLAP technology. This work is done in collaboration with MSA, a programming house using Microsoft technology hence the need for the data to be exported out of Teradata into SQL server (having started out in Oracle from CLWS data entry). See FIG. 14.

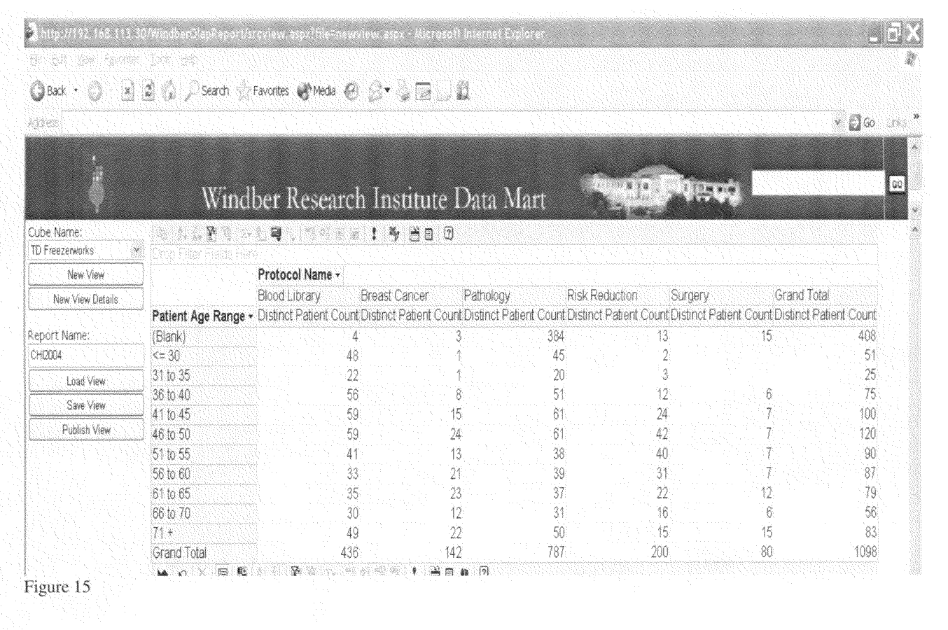

WRI feels that for the ‘Research Gateway’ tool to be useful in the hands of physicians, the reporting needs to be extremely simple to understand, require delivery of no specific software on to the desktop and take under one minute to get to a satisfactory end result. WRI is keen to gather as many user requirements from clinicians as possible. See FIG. 15.

### “Statistical” Data Analysis

A variety of different data analyses underway at WRI fall into the following broad categories:

### Predictive Modeling

At present, Clementine/SPSS is being used to build predictive models of disease progression and outcome. Since the DW is still not truly ‘live’, the models built to date have been largely based on the clinical parameters readily available (sometimes straight out of MS Access) rather than incorporating the data being generated from the high throughput experimental techniques such as gene expression, genetics, proteomics. Approaches currently used include NN, decision trees, SVM, PCA & PLS. We would need to enhance our feature selection and model assessment criteria tuned for biomarker discovery but would be powerful functionality for this expanding area.

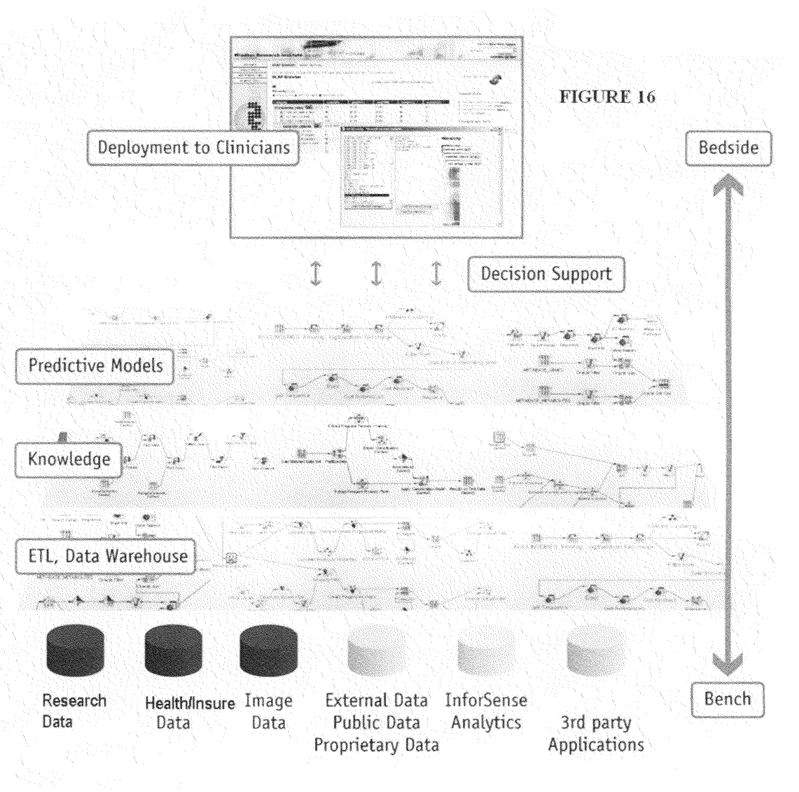

The overall goal is to build these predictive models from the wealth of discovered knowledge and have them power a decision support system that could be deployed out to the physician. See FIG. 16.

### Disease Modeling

WRI is working with a Petri net tool set (modeling methodology tailored for representing and simulating concurrent dynamic systems) from the University of Illinois called Mobius (http://www.mobius.uiuc.edidindex.htmD). Using Petri nets since they can represent system behavior even when the biological mechanism is not fully understood, by combining different levels of abstraction in a single model. Looks pretty powerful system and surprisingly easy to use. Would be useful to integrate with the D W as a source of data for the models maybe using KDE for preprocessing activities.

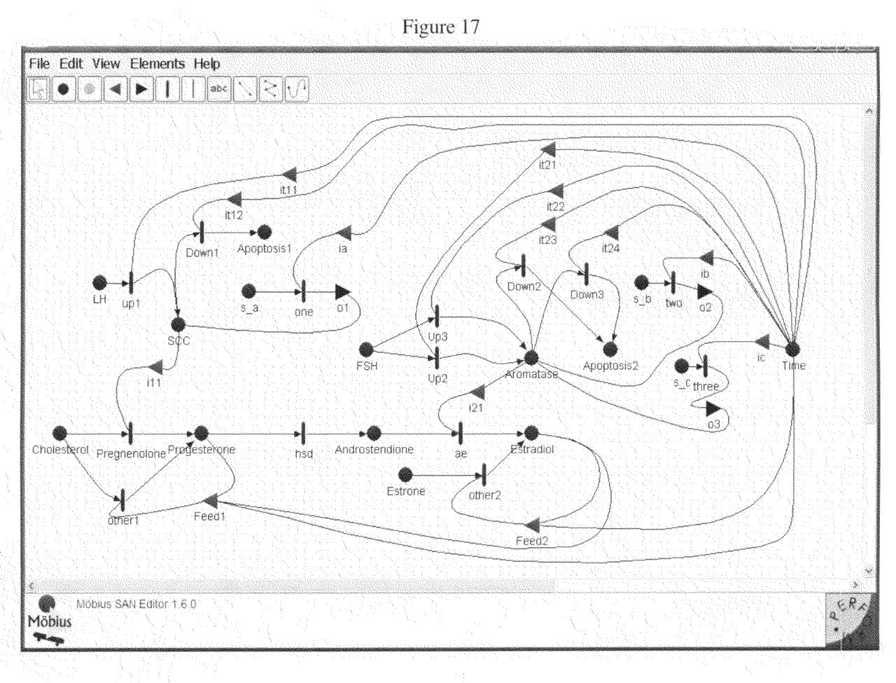

Have their own flavor of Petri nets called Stochastic Activity Networks (SANs) optimized for flow based systems. Modeling a variety of systems using this approach. See FIG. 17.

### Diagnosis Analysis

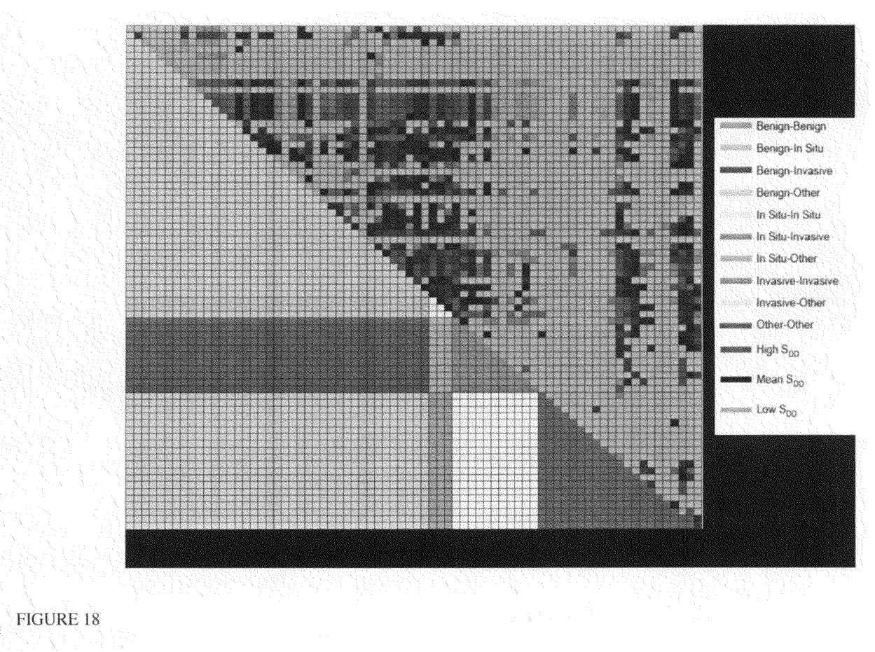

Working on characterizing the heterogeneity in breast cancer tissue by studying patterns in pathology diagnosis. Currently using Clementime/SPSS to study the co-occurrence (frequency based algorithm) of multiple diagnosis terms. Although have recently switched to using R directly which appears much faster if harder to use. Visualizing the output using Spotfire. See FIG. 18

With better sample classification, will be able to more accurately build predictive models from genomic/proteomics data.

IOE with one or two new algorithms could address this area very well linking the DW to the analysis (and Spotfire).

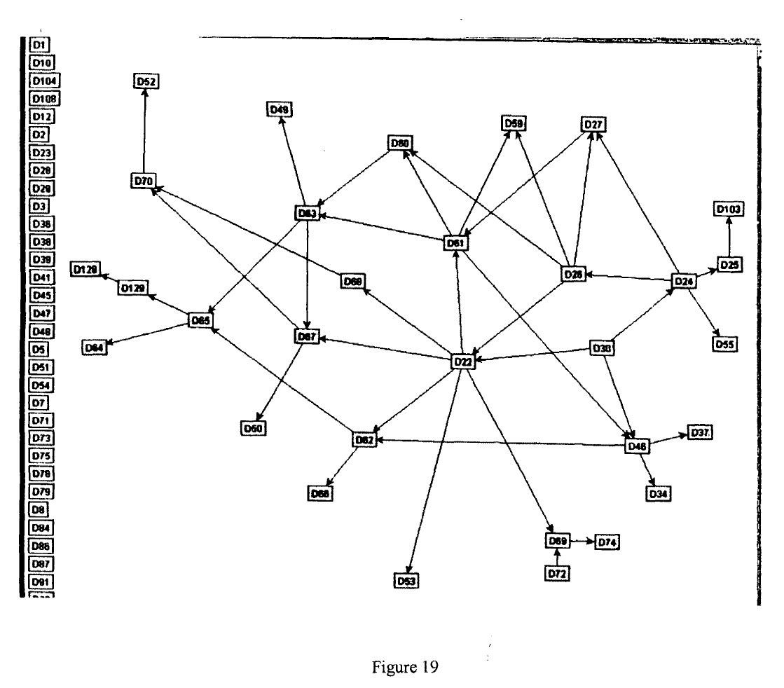

Also, using Bayesian networks on pathology diagnoses to identify independence relationships between diagnoses, and make inferences about the likelihood of a diagnosis based on evidence of other diagnoses. Using software from DecisionQ called FasterAnalytics. See FIG. 19.

### Textmining

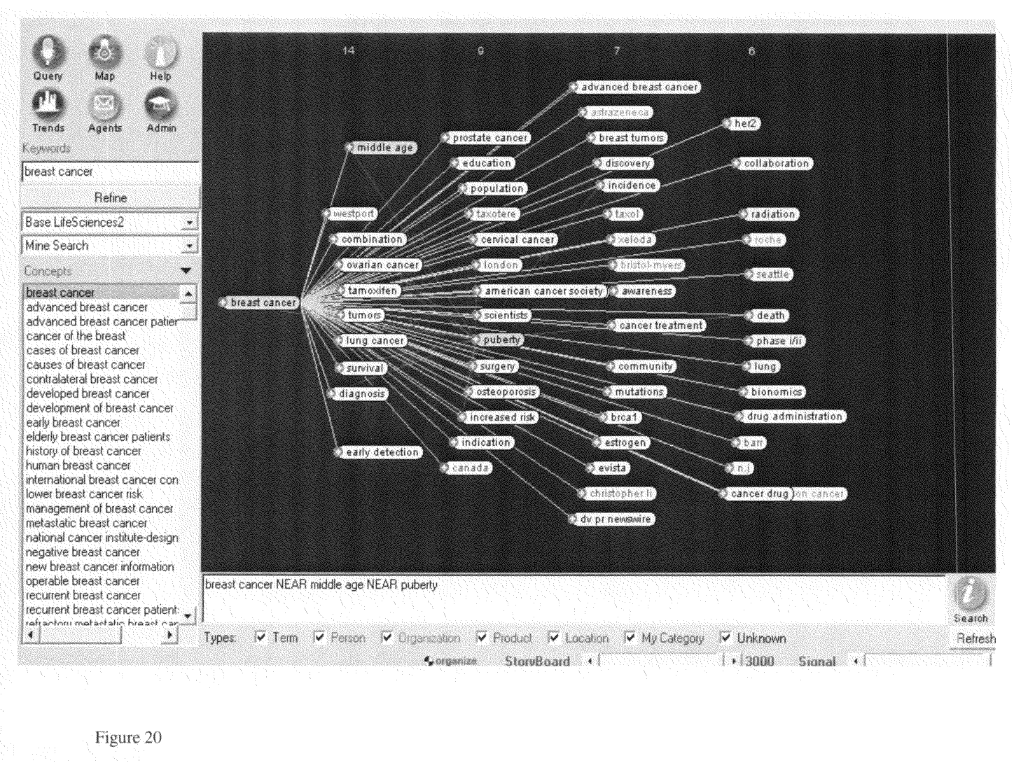

Working on extracting molecular events and changes associated with breast development and breast disease. Major tasks include collection of full text of journal articles, preprocessing of collected text, construction of dictionaries, compilation of patterns, information extraction (NLP) and incorporation of medline information. Currently using LexiMine/SPSS. See FIG. 20.

Although the systems and methods have been described in detail, it will be apparent to those of skill in the art that the systems and methods can be embodied in a variety of specific forms and that various changes, substitutions, and alterations can be made without departing from the spirit and scope of the systems and methods described herein. The described embodiments are only illustrative and not restrictive and the scope of the systems and methods is, therefore, indicated by the following claims. Other embodiments are within the scope of the following claims.

What is claimed is:

1. A method for predicting disease progression or outcome comprising
storing patient information in a database;
storing clinical data in a database;
creating a federated database from at least one database selected from the group consisting of a patient information database, a clinical database, a genomic database, a proteomic database, an imaging database and a disease database; and
submitting a request for information.

2. The method of claim 1, further comprising generating a patient profile with a prediction on disease progression or outcome.

3. The method of claim 1, further comprising generating a treatment plan.

4. The method of claim 1, further comprising predicting disease recurrence.

5. The method of claim 1, further comprising collecting patient information.

6. The method of claim 1, further comprising collecting clinical data.

7. The method of claim 1, wherein the clinical database comprises predicted genetic risk, biomarkers, tumor heterogeneity, pathology report, pathology images, diagnosis co-morbidities, outcomes, diagnostic images, surgical reports, radiation protocols, chemotherapy protocols, post-therapy co-morbidities, protein expression, gene expression, genotyping, sequencing data and DNA copy number analysis from tissue samples or blood samples of the patient or combinations thereof.

8. The method of claim 1, wherein the patient information database comprises clinical history, family history, reproductive history, gynecologic history, lifestyle exposures or quality of life priorities or combinations thereof.

9. The method of claim 1, wherein the genomic database is an Entrez database.

10. The method of claim 1, wherein the proteomic database is an Entrez database.

11. The method of claim 1, wherein the disease is breast cancer.

12. The method of claim 1, wherein the disease is uterine cancer.

13. The method of claim 1, wherein the disease is cervical cancer.

14. The method of claim 1, wherein the disease is endometrial cancer.

15. The method of claim 1, wherein the disease is ovarian cancer.

16. The method of claim 1, wherein the disease is cardiovascular disease.

17. The method of claim 1, wherein the disease is diabetes.

18. The method of claim 1, further comprising creating a federated database from a patient information database.

19. The method of claim 1, further comprising creating a federated database from a clinical database.

20. The method of claim 1, further comprising creating a federated database from a genomic database.

21. The method of claim 1, further comprising creating a federated database from a proteomic database.

22. The method of claim 1, further comprising creating a federated database from an imaging database.

23. The method of claim 1, further comprising creating a federated database from a disease database.

24. A method for diagnosing breast cancer progression or outcome comprising
storing patient information in a database;
storing clinical data in a database;
creating a federated database from at least one database selected from the group consisting of a patient information database, a clinical database, a genomic database, a proteomic database, an imaging database and a disease database; and
submitting a request for information.

25. The method of claim 24, further comprising generating a patient profile with a prediction on breast cancer progression or outcome.

26. The method of claim 24, further comprising generating a treatment plan.

27. The method of claim 24, further comprising predicting disease recurrence.

28. The method of claim 24, further comprising collecting patient information.

29. The method of claim 24, further comprising collecting clinical data.

30. The method of claim 24, wherein the clinical database comprises predicted genetic risk, biomarkers, tumor heterogeneity, pathology report, pathology images, diagnosis co-morbidities, outcomes, diagnostic images, surgical reports, radiation protocols, chemotherapy protocols, post-therapy co-morbidities, protein expression, gene expression, genotyping, sequencing data and DNA copy number analysis from tissue samples or blood samples of the patient or combinations thereof.

31. The method of claim 24, wherein the patient information database comprises clinical history, family history, reproductive history, gynecologic history, lifestyle exposures or quality of life priorities or combinations thereof.

32. The method of claim 24, wherein the genomic database is an Entrez database.

33. The method of claim 24, wherein the proteomic database is an Entrez database.

34. The method of claim 24, further comprising creating a federated database from a patient information database.

35. The method of claim 24, further comprising creating a federated database from a clinical database.

36. The method of claim 24, further comprising creating a federated database from a genomic database.

37. The method of claim 24, further comprising creating a federated database from a proteomic database.

38. The method of claim 24, further comprising creating a federated database from an imaging database.

39. The method of claim 24, further comprising creating a federated database from a disease database.

40. A system for predicting disease progression or outcome comprising a federated database created from at least one database selected from the group consisting of a patient information database, a clinical information database, a genomic database, a proteomic database, an imaging database and a disease database.

41. The system of claim 40, wherein the clinical database comprises predicted genetic risk, biomarkers, tumor heterogeneity, pathology report, pathology images, diagnosis co-morbidities, outcomes, diagnostic images, surgical reports, radiation protocols, chemotherapy protocols, post-therapy co-morbidities, protein expression, gene expression, genotyping, sequencing data and DNA copy number analysis from tissue samples or blood samples of the patient or combinations thereof.

42. The system of claim 40, wherein the patient information database comprises clinical history, family history, reproductive history, gynecologic history, lifestyle exposures or quality of life priorities or combinations thereof.

43. The system of claim 40, wherein the genomic database is an Entrez database.

44. The system of claim 40, wherein the proteomic database is an Entrez database.
